(19) United States
(12) Patent Application Publication
(10) Pub. No.: US 2013/0211328 A1
(43) Pub. Date: Aug. 15, 2013

(54) DIFFERENTIATION FOR A DRUG DELIVERY DEVICE

(71) Applicants: David Plumptre, Worcestershire (GB); Richard James Vincent Avery, Gloucestershire (GB)

(72) Inventors: David Plumptre, Worcestershire (GB); Richard James Vincent Avery, Gloucestershire (GB)

(73) Assignee: SANOFI-AVENTIS DEUTSCHLAND GMBH, Frankfurt am Main (DE)

(21) Appl. No.: 13/810,501

(22) PCT Filed: Aug. 11, 2011
(86) PCT No.: PCT/EP11/63847

§ 371 Date: Apr. 26, 2013

Related U.S. Application Data
(60) Provisional application No. 61/373,387, filed on Aug. 13, 2010.

(30) Foreign Application Priority Data
Oct. 26, 2010 (EP) 10188855.0

(51) Int. Cl. A61M 5/50

(52) U.S. Cl. CPC A61M 5/5086
USPC 604/111

(57) ABSTRACT

A drug delivery device with differentiating features is provided. The drug delivery device comprises a dose setting mechanism, a cartridge holder, and a cartridge contained within the cartridge holder. The cartridge holder is secured to the dose setting mechanism and comprises a collar or ring. The ring has the same color as other differentiation features on either the dose setting mechanism or the cartridge, or both. The color-coded ring is configured on the cartridge holder such that when the cartridge holder is assembled with the dose setting mechanism, the color-coded ring remains visible.

### CROSS REFERENCE TO RELATED APPLICATIONS

The present application is a U.S. National Phase Application pursuant to 35 U.S.C. §371 of International Application No. PCT/EP2011/063847 filed Aug. 11, 2011, which claims priority to U.S. Provisional Patent Application No. 61/373,387 filed Aug. 13, 2010 and European Patent Application No. 10188855.0 filed Oct. 26, 2010. The entire disclosure contents of these applications are herewith incorporated by reference into the present application.

### TECHNICAL FIELD

The present patent application is generally directed to reservoirs, particularly reservoirs containing a medicament. More particularly, the present application is generally directed to mechanisms for differentiation for use with a reservoir and a reservoir holder so as to allow a user or healthcare provider to quickly and easily differentiate various drug delivery devices along with their various components.

### BACKGROUND

Such differentiation mechanisms may also include either coding on the cartridge, coding on the cartridge holder, and/or coding on the dose setting mechanism. Such coding may comprise mechanical and/or electronic coding. As just one example, such medicament reservoirs may comprise an ampoule, a cartridge, a vial, or a pouch, and may be used with a medical delivery device. Exemplary medical or drug delivery devices include, but are not limited to syringes, pen type injection syringes, pumps, inhalers, or other similar injection or infusing devices that require at least one reservoir containing at least one medicament.

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge is empty, the user is recommended to dispose of the entire device.

Such known self administration systems requiring the removal and reloading of empty cartridges have certain limitations. For example, in certain generally known systems, a user simply loads a new cartridge into the delivery system without the drug delivery device or without the cartridge having any mechanism of preventing removal and subsequent cross use of an incorrect cartridge. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long insulin could result in injury or even death.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

A number of solutions could ensure that an injection device or a drug delivery device is properly paired with its drug type so as to prevent unwanted cartridge cross use, for example education, differentiation, and dedication. For example, education might include training sessions, or instruction leaflets. As used herein, the term differentiation is preferably used to indicate a type of medicament to the user. The differentiation may be effected by e.g. text, color, and/or tactile features. As used herein, the term dedication or coding is preferably used to mean the desired cooperation or linkage between various components of a drug delivery device such that only correct combinations are allowed. As just one example, such coding may include mechanical and/or electronic coding. In the case of mechanical coding, the coded components, if two correct components are combined, may be adapted to cooperate mechanically to allow a combination, e.g. a connection, of the two components. If two incorrect components are intended to be combined, e.g. connected, the coded components may be adapted to mechanically cooperate to prevent a combination, e.g. a connection, of the two components. Mechanical coding may be achieved by mechanical coding features with which the components are provided and which are adjusted to each other such that they may cooperate if a combination should be allowed or not if a combination should be prevented. In the case of electronic coding, e.g. a sensor may detect a marking on the coded component and an electronic control unit, e.g. comprising a processor, may determine whether this marking indicates that the marked component has the right property for the purpose it should serve or not. If the component has the wrong properties a combination may be prevented by a mechanism triggered by the processor or by an alarm preventing the user from trying to combine, e.g. assemble, this component with the remaining component(s). A combination of all the above solutions can be more effective than any single solution.

Cartridges, such as cartridges containing a medicament, may be marked with details of the medicament that they contain, and may be differentiated with a specific color for that medicament. Existing disposable pen type syringes have colored bodies, labels, and/or other indication means that match the medicament differentiation colors. Certain existing reusable pen type syringes are available in several colors, but generally not in medicament differentiation colors. Although the cartridge itself may be marked with details of the medicament that cartridge contains, this marking may be difficult to see or to properly identify after the cartridge is loaded into the drug delivery device. As such, a user may select an incorrect drug delivery device containing an incorrect cartridge and thereby dispense an incorrect medicament.

It would therefore be advantageous for a drug delivery device or system, in particular for a reusable device or system, to comprise some type of marking or indication comprising a medicament differentiation, such as a medicament differentiation color. However, with a differentiated device, e.g. a reusable device, there is a risk that the user might load a cartridge in the wrong device, and then rely on the differentiation on the device rather than reading the cartridge label, and so dispense the wrong medicament. Alternatively, the various detachable parts of the reusable drug delivery device might be dedicated or coded (mechanically and/or electrically) to each other, but without any differentiation. For example, the cartridge might be coded to the cartridge holder, the cartridge holder might be coded to the dose setting mechanism, and/or the cap might be coded to the cartridge holder and/or dose setting mechanism. The risk of a fully dedicated device is that it might be difficult to understand, so the user might apply excessive force to assemble the device, and damage the coding so that a cartridge fits into an incorrect device. A solution would be a fully differentiated and dedicated drug delivery device, where parts of the device are differentiated for the medicament, and dedicated (or coded) to one another.

Although a differentiated and dedicated drug delivery device should increase user safety, it may be expensive to develop and distribute a different device for each medicament. In one of the presently proposed systems and methods of drug delivery device differentiation, the proposed differentiation and/or dedication may be applied to detachable parts. Preferably, the proposed differentiation and/or dedication may only be applied to a small number of detachable parts. Preferably, the differentiation on the detachable parts is visible even when the drug delivery device is assembled. As such, the dose setting mechanism does not need differentiation for each type of medicament. Therefore the same dose setting mechanism can be used for a range of medicaments. In one arrangement, the same dose setting mechanism may be used for drugs with the same concentration as each other.

### SUMMARY

It is an object of the present disclosure to provide one or more means which facilitate provision of a novel drug delivery device or novel elements for a drug delivery device. In particular, it is intended to provide one or more means which facilitate overcoming one, more or all of the problems described herein.

This object is achieved, for example, by the subject matter of the independent claim. Advantageous refinements are the subject matter of the dependent claims. As it is readily apparent from the disclosure below, various ways to provide for novel elements for drug delivery devices are disclosed herein. Accordingly, the disclosure may cover solutions which are different from the ones currently claimed.

One aspect relates to an assembly or an arrangement comprising at least one of or both of a cartridge and a cartridge holder, wherein at least one of or both of the cartridge and the cartridge holder is provided with a differentiation feature.

The cartridge may be assembled or retained in the cartridge holder. The cartridge may comprise drug or medicament. The cartridge or the cartridge holder may be configured to be assembled to a drug delivery device in particular to a dose setting mechanism of a drug delivery device. The cartridge may be retained within the holder and the holder with the cartridge in it may be assembled to the drug delivery device. Alternatively, a separate cartridge holder can be dispensed with. In this case, the cartridge itself may be immediately assembled to the drug delivery device. The cartridge may be a molded cartridge.

In one embodiment, the differentiation feature is arranged to remain visible when said cartridge or cartridge holder is assembled to the dose setting mechanism of the drug delivery device. The differentiation feature may be arranged near or at the proximal end of the cartridge or the cartridge holder.

Another aspect relates to a drug delivery system. The drug delivery system may comprise a drug delivery device. The drug delivery device may comprise a dose setting mechanism. The cartridge holder may comprise or may be provided with a collar. The cartridge holder may be secured to the dose setting mechanism. The cartridge may be contained in the cartridge holder. The cartridge may comprise a differentiation feature. When the cartridge holder is assembled with said drug delivery device, the collar expediently remains visible. The differentiation feature provided on the cartridge may comprise a color. The collar may comprise a differentiation feature, in particular a differentiation feature having the same or a similar color as a differentiation feature provided on said cartridge. The collar may wrap around an exterior circumference of said cartridge holder, preferably around the entire circumference.

The drug delivery system may comprise an assembly or an arrangement as described further above and below. Features described in connection with the cartridge, the cartridge holder, the assembly or the arrangement may thus also apply to the drug delivery system and vice versa.

In one embodiment, the differentiation feature comprises a color. When the differentiation feature is provided on the cartridge holder at least a portion of said cartridge may comprise the same color as said differentiation feature and vice versa.

In one embodiment, a main body is affixed to a proximal end or near the proximal end of said cartridge or cartridge holder. The main body may comprise a ring or collar. Accordingly, a ring or collar may be affixed to or near the proximal end of the cartridge or the cartridge holder. The differentiation feature may be provided along said main body. The ring or collar may be color-coded. The ring or collar may wrap around an exterior circumference of said cartridge or cartridge holder.

The proximal end of the drug delivery system, the device or an element thereof may be that end which is or is to be arranged furthest away from a distal end of the drug delivery system or device. The distal end of the drug delivery system, the device or an element thereof may be that end which is or is to be arranged closest to the dispensing end of the device or system.

In one embodiment, a cap is provided. The cap may be provided for covering at least a distal end of said cartridge or said cartridge holder. The cap may comprise a clip. The cap may be removably affixable to the cartridge, the cartridge holder, the drug delivery device or the drug delivery system. When said cap is removably affixed to the applicable element mentioned above, said differentiation feature expediently remains visible. In particular, when the cap is placed over said cartridge holder, a proximal end of said cap may abut the collar, and the collar expediently remains visible. The cap may comprise the same color as one of or all of the differentiation features. In particular, the cap may comprise the same color as the differentiation feature provided on the cartridge holder and/or as the differentiation feature on the cartridge. The cap may comprise the same color as the ring or collar.

In one embodiment, the cartridge is coded, e.g. mechanically coded, to ensure or to help ensure that the cartridge can only be assembled into a correct cartridge holder. For example, if there are two cartridge holders, the cartridge may be assembled into one of them, whereas assembling the cartridge into the other holder may be prevented, e.g. by mechanical interaction of respective coding features.

In one embodiment, the cartridge holder comprises an interior surface. The cartridge holder may comprise an aperture defined by said interior surface. The interior surface may comprise a groove. The dose setting mechanism may comprise a corresponding protrusion. When said dose setting mechanism is inserted into said cartridge holder aperture, said protrusion may be arranged to slide within said groove.

In one embodiment, the cap is coded, e.g. mechanically coded, to help ensure or to ensure that said cap can only be assembled onto a correct dose setting mechanism, cartridge, or cartridge holder. This coding may be achieved in a way analogue to the coding between cartridge and cartridge holder.

In one embodiment, the cartridge holder is coded, e.g. mechanically coded to help ensure or to ensure that said cartridge holder can only be assembled onto a correct dose setting mechanism. This coding may be achieved in a way analogue to the coding between cartridge and cartridge holder.

In one embodiment, the cartridge holder and said dose setting mechanism further comprise a fastening mechanism wherein said fastening mechanism couples said cartridge holder to said dose setting mechanism.

In one embodiment, the cartridge is removably contained within said cartridge holder.

In one embodiment, the cartridge is non-removably contained within said cartridge holder.

In one embodiment, the drug delivery device comprises a reusable drug delivery device.

In one embodiment, the drug delivery device comprises a disposable drug delivery device.

According to an exemplary arrangement, a differentiation feature for a cartridge holder within a drug delivery device is provided. The differentiation feature comprises a main body that is affixed to the proximal end of the cartridge holder. At least a portion of the main body comprises a color portion. This colored portion is visible after insertion of a cartridge into the cartridge holder and after the cartridge holder is assembled within a drug delivery device.

In another alternative arrangement, a collar on a cartridge holder is provided. The collar comprises a main body with an exterior surface. The main body is affixed to a proximal end of the cartridge holder, and the exterior surface comprises a differentiation feature. When the cartridge holder is assembled within a drug delivery device, the differentiation feature remains visible.

In yet another arrangement, a drug delivery system is provided. The drug delivery system comprises a dose setting mechanism, a cartridge holder, and a cartridge. The cartridge holder is secured to the dose setting mechanism. A cartridge with a differentiation feature is contained within the cartridge holder. The cartridge holder comprises a collar. The collar has the same color as the differentiation feature, and when the cartridge holder is assembled within a drug delivery device, the collar remains visible. Alternatively or in addition, the differentiation may be provided on a cap for the drug delivery device.

The term “drug”, “medication” or “medicament”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36[Asp28] Exendin-4(1-39),

### des Pro36[IsoAsp28] Exendin-4(1-39),

### des Pro36[Met(O)14, Asp28] Exendin-4(1-39),

### des Pro36[Met(O)14, IsoAsp28] Exendin-4(1-39),

### des Pro36[Trp(O2)25, Asp28] Exendin-4(1-39),

### des Pro36[Trp(O2)25, IsoAsp28] Exendin-4(1-39),

### des Pro36[Met(O)14 Trp(O2)25, Asp28] Exendin-4(1-39),

### des Pro36[Met(O)14 Trp(O2)25, IsoAsp28] Exendin-4(1-39); or

### des Pro36[Asp28] Exendin-4(1-39),

### des Pro36[IsoAsp28] Exendin-4(1-39),

### des Pro36[Met(O)14, Asp28] Exendin-4(1-39),

### des Pro36[Met(O)14, IsoAsp28] Exendin-4(1-39),

### des Pro36[Trp(O2)25, Asp28] Exendin-4(1-39),

### des Pro36[Trp(O2)25, IsoAsp28] Exendin-4(1-39),

### des Pro36[Met(O)14 Trp(O2)25, Asp28] Exendin-4(1-39),

### des Pro36[Met(O)14 Trp(O2)25, IsoAsp28] Exendin-4(1-39),

wherein the group -Lys6-NH2 may be bound to the C-terminus of the Exendin-4 derivative; or an Exendin-4 derivative of the sequence

### H-(Lys)6-des Pro36[Asp28] Exendin-4(1-39)-Lys6-NH2,

### des Asp28 Pro36, Pro37, Pro38Exendin-4(1-39)-NH2,

### H-(Lys)6-des Pro36, Pro38[Asp28] Exendin-4(1-39)-NH2,

### H-Asn-(Glu)5des Pro36, Pro37, Pro38[Asp28] Exendin-4(1-39)-NH2,

### des Pro36, Pro37, Pro38[Asp28] Exendin-4(1-39)-(Lys)6-NH2,

### H-(Lys)6-des Pro36, Pro37, Pro38[Asp28] Exendin-4(1-39)-(Lys)6-NH2,

### H-Asn-(Glu)5-des Pro36, Pro37, Pro38[Asp28] Exendin-4(1-39)-(Lys)6-NH2,

### H-(Lys)6-des Pro36[Trp(O2)25, Asp28] Exendin-4(1-39)-Lys6-NH2,

### H-des Asp28 Pro36, Pro37, Pro38[Trp(O2)25] Exendin-4(1-39)-NH2,

### H-(Lys)6-des Pro36, Pro37, Pro38[Trp(O2)25, Asp28] Exendin-4(1-39)-NH2,

### H-Asn-(Glu)5-des Pro36, Pro37, Pro38[Trp(O2)25, Asp28] Exendin-4(1-39)-NH2,

### des Pro36, Pro37, Pro38[Trp(O2)25, Asp28] Exendin-4(1-39)-(Lys)6-NH2,

### H-(Lys)6-des Pro36, Pro37, Pro38[Trp(O2)25, Asp28] Exendin-4(1-39)-(Lys)6-NH2,

### H-Asn-(Glu)5-des Pro36, Pro37, Pro38[Trp(O2)25, Asp28] Exendin-4(1-39)-(Lys)6-NH2,

### H-(Lys)6-des Pro36[Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-des Pro36, Pro37, Pro38[Met(O)14, Asp28] Exendin-4(1-39)-NH2,

### H-Asn-(Glu)5-des Pro36, Pro37, Pro38[Met(O)14, Asp28] Exendin-4(1-39)-NH2,

### des Pro36, Pro37, Pro38[Met(O)14, Asp28] Exendin-4(1-39)-(Lys)6-NH2,

### H-(Lys)6-des Pro36, Pro37, Pro38[Met(O)14, Asp28] Exendin-4(1-39)-(Lys)6-NH2,

### H-Asn-(Glu)5des Pro36, Pro37, Pro38[Met(O)14, Asp28] Exendin-4(1-39)-(Lys)6-NH2,

### H-Lys6-des Pro36[Met(O)14, Trp(O2)25, Asp28] Exendin-4(1-39)-Lys6-NH2,

### H-des Asp28 Pro36, Pro37, Pro38[Met(O)14, Trp(O2)25] Exendin-4(1-39)-NH2,

### H-(Lys)6-des Pro36, Pro37, Pro38[Met(O)14, Asp28] Exendin-4(1-39)-NH2,

### H-Asn-(Glu)5-des Pro36, Pro37, Pro38[Met(O)14, Trp(O2)25, Asp28] Exendin-4(1-39)-NH2,

### des Pro36, Pro37, Pro38[Met(O)14, Trp(O2)25, Asp28] Exendin-4(1-39)-(Lys)6-NH2,

### H-(Lys)6-des Pro36, Pro37, Pro38[Met(O)14, Trp(O2)25, Asp28] Exendin-4(S1-39)-(Lys)6-NH2,

### H-Asn-(Glu)5-des Pro36, Pro37, Pro38[Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

In the following text, a set of particularly advantageous aspects is specified. Within the set, numbers are used to identify specific aspects. This facilitates referencing the features of specific aspects in other aspects.

1. A cartridge holder, said cartridge holder comprising:

a main body affixed to a proximal end of said cartridge holder,

a differentiation feature is provided along said main body,

wherein said differentiation feature remains visible when said cartridge holder is assembled to a dose setting mechanism of a drug delivery device.

2. The subject matter of aspect 1 further comprising a cap for covering at least a distal end of said cartridge holder, wherein when said cap is removably affixed to said cartridge holder, said differentiation feature remains visible.

3. The subject matter of aspect 1 wherein the differentiation feature comprises a color.

4. The subject matter of aspect 3 wherein at least a portion of said cartridge comprises a same color as said colored portion on said main body.

5. The subject matter of aspect 1 wherein said cartridge holder and said dose setting mechanism further comprise a fastening mechanism

wherein said fastening mechanism couples said cartridge holder to said dose setting mechanism.

6. The subject matter of aspect 1 wherein said cartridge holder comprises an interior surface and an aperture defined by said interior surface.

7. The subject matter of aspect 6 wherein said interior surface comprises a groove.

8. The subject matter of aspect 7 wherein said dose setting mechanism comprises a corresponding protrusion,

wherein when said dose setting mechanism is inserted into said cartridge holder aperture, said protrusion slides within said groove.

9. The subject matter of aspect 1, wherein said main body comprises a color-coded ring.

10. The subject matter of aspect 9 wherein said color-coded ring wraps around an exterior circumference of said cartridge holder.

11. The subject matter of aspect 1 wherein said drug delivery device comprises a reusable drug delivery device.

12. The subject matter of aspect 1 wherein said cartridge is mechanically coded to help ensure that the cartridge can only be assembled into a correct cartridge holder.

13. The subject matter of aspect 1 wherein said cartridge holder is coded so as to help ensure that said cartridge holder can only be assembled onto a correct dose setting mechanism.

14. The subject matter of aspect 1 wherein said cartridge holder comprises a molded cartridge.

15. A collar for use with a cartridge holder, said collar comprising:

a main body with an exterior surface, wherein said main body is affixed near a proximal end of a cartridge holder,

wherein said exterior surface of said main body comprises a differentiation feature

such that said differentiation feature remains visible after said cartridge holder and a dose setting member are assembled.

16. The subject matter of aspect 15 further comprising a cap for covering at least a distal end of said cartridge holder, wherein when said cap is removably affixed to said cartridge holder, said differentiation feature remains visible.

17. The subject matter of aspect 15 wherein said cartridge is mechanically coded to help ensure that said cartridge can only be assembled into a correct cartridge holder.

18. A drug delivery system comprising:

a drug delivery device comprising a dose setting mechanism;

a cartridge holder comprising a collar, wherein said cartridge holder is secured to said dose setting mechanism; and

a cartridge contained within said cartridge holder, said cartridge comprising a differentiation feature,

wherein when said cartridge holder is assembled with said drug delivery device, said collar remains visible.

19. The subject matter of aspect 18 wherein said collar comprises a differentiation feature having a similar color as said differentiation feature provided on said cartridge.

20. The subject matter of aspect 18 wherein said collar is a color-coded ring.

21. The subject matter of aspect 18 wherein said cartridge holder and said dose setting mechanism further comprise a fastening mechanism

wherein said fastening mechanism couples said cartridge holder to said dose setting mechanism.

22. The subject matter of aspect 18 wherein said cartridge holder comprises an interior surface and an aperture defined by said interior surface.

23. The subject matter of aspect 22, wherein said interior surface comprises a groove.

24. The subject matter of aspect 23, wherein said dose setting mechanism comprises a corresponding protrusion, and wherein when said dose setting mechanism is inserted into said cartridge holder aperture, said protrusion slides within said groove.

25. The subject matter of aspect 18 wherein said collar wraps around the entire exterior circumference of said cartridge holder.

26. The subject matter of aspect 18 further comprising a cap, wherein said cap comprises a clip.

27. The subject matter of aspect 26 where said cap is coded to help ensure that said cap can only be assembled onto a correct dose setting mechanism.

28. The subject matter of aspect 26 wherein said cap comprises the same color as said collar.

29. The subject matter of aspect 26 wherein when said cap is placed over said cartridge holder, a proximal end of said cap abuts said collar, and said collar remains visible.

30. The subject matter of aspect 18 wherein said cartridge is removably contained within said cartridge holder.

31. The subject matter of aspect 18 wherein said cartridge is non-removably contained within said cartridge holder.

32. The subject matter of aspect 18 wherein said drug delivery device comprises a reusable drug delivery device.

33. The subject matter of aspect 18 wherein said cartridge is mechanically coded to ensure that said cartridge can only be assembled into a correct cartridge holder.

34. The subject matter of aspect 18 wherein said cartridge holder is coded to help ensure that said cartridge holder can only be assembled onto a correct dose setting mechanism.

Features described herein-above and below in conjunction with different aspects, embodiments, arrangements etc., may, of course, be combined with each other.

### BRIEF DESCRIPTION OF THE DRAWINGS

These as well as other advantages of various aspects of the present disclosure will become apparent to those of ordinary skill in the art by reading the following detailed description, with appropriate reference to the accompanying drawings.

Exemplary embodiments are described herein with reference to the drawings, in which:

FIG. 1 illustrates an exemplary pen type drug delivery device;

FIG. 2 illustrates a cartridge that may be loaded into a cartridge holder of the pen type drug delivery device illustrated in FIG. 1;

FIG. 3 illustrates a first arrangement of a differentiation feature on a cartridge holder that may be used with a pen type drug delivery device such as the drug delivery device illustrated in FIG. 1;

FIG. 4 illustrates the differentiation feature illustrated in FIG. 3 as a cartridge is inserted within the cartridge holder;

FIG. 5 illustrates the differentiation feature on the cartridge holder with the cartridge in FIG. 4 inserted, and a dose setting mechanism;

FIG. 6 illustrates the drug delivery device in FIG. 5 in the assembled position;

FIG. 7 illustrates the drug delivery device in FIG. 6 with a cap; and

FIG. 8 illustrates the drug delivery device in FIG. 7 with the cap fully assembled on the cartridge holder.

### DETAILED DESCRIPTION

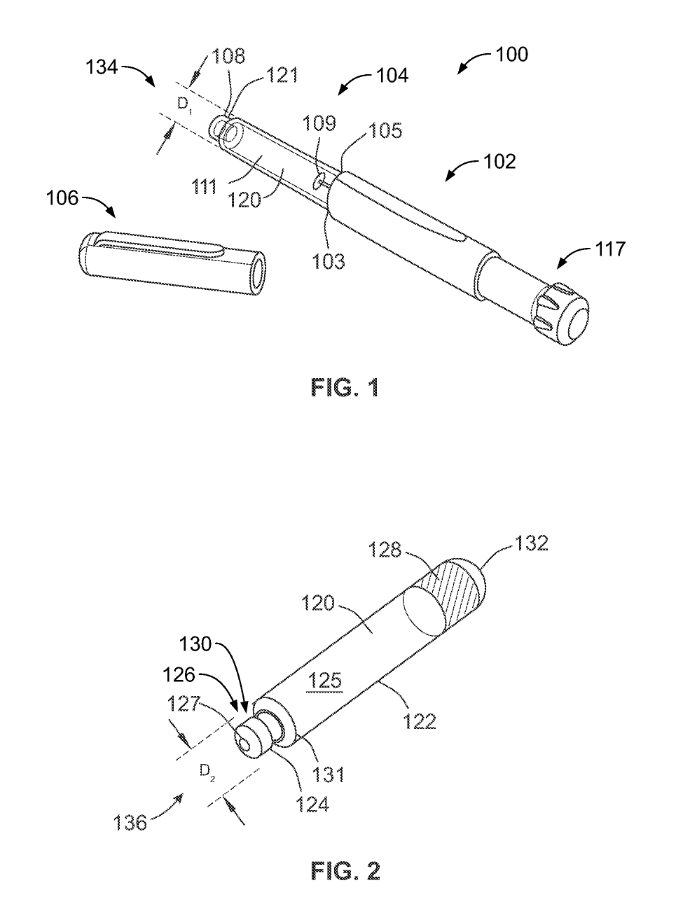

Referring to FIG. 1, there is shown a drug delivery device 100 in the form of a pen type syringe. This drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The dose setting mechanism 102 comprises a piston rod 109, such as a threaded piston rod that rotates when a dose is injected.

To inject a previously set dose, a double ended needle assembly is attached to a distal end 108 of the cartridge holder. Preferably, the distal end of the holder comprises a thread 121 (or other suitable connecting mechanism such as a snap lock, snap fit, form fit, or bayonet lock mechanism) so that the needle assembly may be removably attached to the distal end of the holder. When the drug delivery device is not in use, the removable cap 106 can be releasably retained over the cartridge holder 104.

An inner cartridge cavity 111 defined by the cartridge holder 104 is dimensioned and configured to securely receive and retain the cartridge 120. FIG. 2 illustrates a perspective view of the cartridge 120 that may be used with the drug delivery device illustrated in FIG. 1. The cartridge 120 includes a generally tubular barrel 122 extending from a distal end 130 to a proximal end 132. The distal end 130 is defined by an inwardly converging shoulder 131.

At the distal end 130, the cartridge 120 includes a smaller diameter neck 126 and this neck projects distally from the shoulder 131 of the barrel 122. Preferably, this smaller diameter neck 126 is provided with a large diameter annular bead (not shown) and this bead extends circumferentially thereabout at the extreme distal end of the neck 126. A pierceable seal or septum 127 is securely mounted across the open distal end defined by the neck. The seal 127 may be held in place by a metallic sleeve or ferrule 124. This ferrule 124 may be crimped around the circumferential bead at the distal end of the neck. The diameter of ferrule 124 is shown by D2 136. The medicament 125 is pre-filled into the cartridge 120 and is retained within the cartridge, in part, by the pierceable seal 127, the metallic sleeve 124, and the stopper 128. The stopper 128 is in sliding fluid-tight engagement with the inner tubular wall of the barrel 122. Axially directed forces acting upon the stopper 128 during dose injection or dose administration urges the medication 125 from the cartridge though a double ended needle mounted onto the distal end 130 of the cartridge holder 104 and into the injection site. Such axial forces may be provided by the piston rod 109.

A portion of the cartridge holder 104 defining the cartridge holder cavity 111 is of substantially uniform diameter represented in FIG. 1 by D1 134. This diameter D1 is preferably slightly greater than the diameter D2 of the cartridge 120. The interior of the cartridge holder includes an inwardly-extending annual portion or stop that is dimensioned to prevent the cartridge 120 from moving within the cartridge holder 104. In this manner, when the cartridge 120 is loaded into the cavity 111 of the cartridge holder 104 and the cartridge holder 104 is then connected to the dose setting member 102, the cartridge 120 will be securely held within the cartridge cavity. More particularly, the neck 126 and ferrule 124 of the cartridge 120 are inserted in a proximal to distal direction into the open proximal end of the cartridge holder 104 with the ferrule eventually passing entirely into the holder 104. With the holder 104 removably coupled to the dose setting mechanism 102, the proximal end of the cartridge 120 will typically abut a stop provided by the dose setting member 102.

A number of doses of a medicament 125 may be dispensed from the cartridge 120. Preferably, the cartridge 120 contains a type of medicament that must be administered often, such as one or more times a day. One such medicament is insulin. The stopper 128 is retained in a first end or proximal end of the cartridge 120 and receives an axial force created by the piston rod 109 of the dose setting mechanism 102.

The dose setting mechanism 102 comprises a dose setter 117 at the proximal end of the dose setting mechanism. In one preferred arrangement, the dose setter 117 is rotated to set a dose. To administer this set dose, the user attaches the needle assembly comprising a double ended needle on the distal end of the cartridge holder. In this manner, the needle assembly pierces the seal 127 of the cartridge 120 and is therefore in liquid communication with the medicament 125. The user pushes on the dose setter 117 to inject the set dose. The same dose setting and dose administration procedure is followed until the medicament 125 in the cartridge is expended, at which time a new cartridge may be loaded in the device. To exchange an empty cartridge, the user is called upon to remove the cartridge holder 104 from the dose setting mechanism 102.

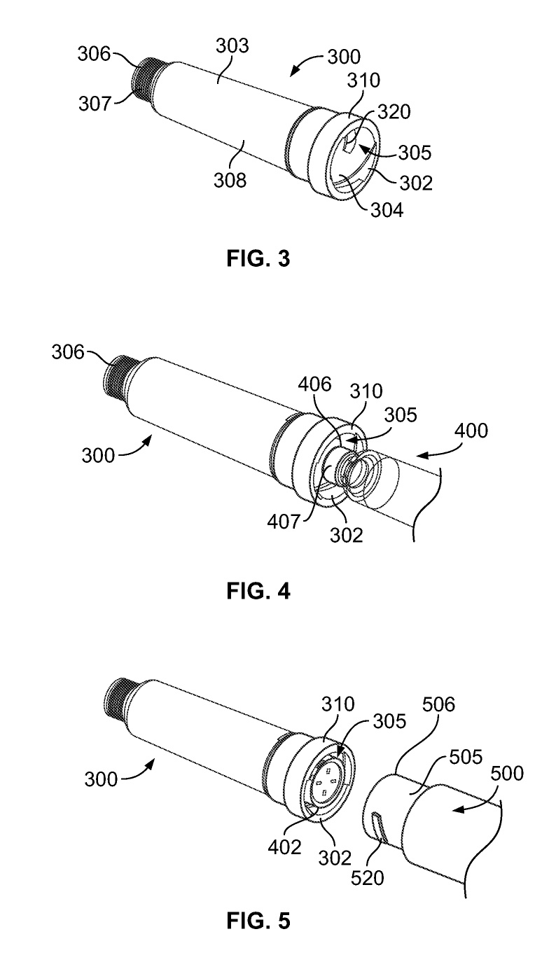

FIG. 3 illustrates a first arrangement of a differentiation feature on a cartridge holder that may be used with a pen type drug delivery device such as the drug delivery device illustrated in FIG. 1. More specifically, a differentiation feature in the form of a color-coded ring 310 is present on a cartridge holder 300. Alternatively, this differentiation feature may comprise text, an image or images, or other tactile features. The differentiation may be applied in a number of ways. For example, it may printed onto the surface of the cartridge holder, or over molded around the cartridge holder. Alternatively, it may be formed as a separate part that is preferably non-detachably retained on the cartridge holder. Color-coded ring 310 is preferably located at the proximal end 302 of the cartridge holder 300. In this manner, and as explained in greater detail below, color-coded ring 310 can provide a differentiation feature to the drug delivery device during both assembly and for selection by a user after assembly.

The cartridge holder 300 shown in FIG. 3 comprises a cylindrically shaped main body 303 with an interior surface 304 defining a centrally located aperture 305. Preferably, aperture 305 has a slightly larger diameter than the diameter of a cartridge. Cartridge holder 300 also comprises an exterior surface 308 and a distal end 306. Distal end 306 preferably comprises a thread 307 (or other suitable connecting mechanism such as a snap lock, snap fit, form fit, or bayonet lock mechanism) so that a needle assembly may be removably attached to distal end 306.

Cartridge holder 300 may contain a 3 ml cartridge, which may be either detachable or non-detachable. Cartridge holder 300 may also comprise mechanical coding to help ensure that a cartridge can only be fitted in the correct cartridge holder. FIG. 3 shows an exemplary fastening means 320 that may also operate as a mechanical coding to help ensure that a cartridge holder can only be fitted to a correct dose setting mechanism. The same dose setting mechanism may be used for a range of medicaments, for example, a range of medicaments having the same concentration. Each medicament within the range would share the same geometry of fastening means, but each range of medicaments would have a different fastening means. As illustrated, fastening means 320 comprises a groove that allows for axial, helical, and then rotational travel. However, the fastening means 320 may be any fastening means and may comprise any combination of directions of travel, including purely one direction of travel. The preferable location for the fastening means 320 is on the interior of the cartridge holder. Such a fastening means 320 location may be preferable as it allows the differentiation to overlap with the fastening means in the axial direction. It therefore has an advantage of reducing the overall length of the cartridge and cartridge holder while providing for the full dispensable volume of a 3 ml cartridge to fit within cartridge holder 300. This also provides for the full dispensable volume of a 3 ml cartridge to be visible within cartridge holder 300. However, a fastening means may be located in another place along the interior surface 304 or exterior surface of cartridge holder 300. In addition to or in place of fastening means 320, cartridge holder 300 may comprise a plurality of other coding features.

FIG. 4 shows the cartridge holder 300 with color-coded ring 310 illustrated in FIG. 3, as a cartridge 400 is inserted into cartridge holder 300. In FIG. 4, a distal end 406 of cartridge 400 is inserted into aperture 305 at the proximal end 302 of cartridge holder 300. Distal end 406 of cartridge 400 may comprise a designated color 407. For example, in one arrangement, this color may be provided on a ferrule of the cartridge, but may be applied in a different location. Designated color 407 may be the same color as color-coded ring 310 so that a user can easily determine that the proper cartridge is being inserted into its corresponding cartridge holder.

Cartridge 400 is then pushed inside aperture 305 until it is fully inserted into cartridge holder 300, as shown in FIG. 5. In FIG. 5, proximal end 402 of cartridge 400 resides within aperture 305, such that proximal end 302 of cartridge holder 300 extends beyond proximal end 402. In an alternative configuration, proximal end 402 of cartridge 400 may protrude from proximal end 302 of cartridge holder 300, allowing the user to rotate cartridge 400 to properly align the coding on the cartridge and the cartridge holder.

In an alternative configuration, no cartridge holder may be present, and instead cartridge holder 300 may comprise a molded cartridge or the cartridge may be a molded cartridge.

FIG. 5 also shows the distal end 506 of a dose setting mechanism 500. As shown in FIG. 5, toward distal end 506 the dose setting mechanism comprises a narrow section 505. Narrow section 505 is sized such that the narrow section fits over cartridge 400 yet within aperture 305 of cartridge holder 300. Narrow section 505 may also comprise, on its exterior surface, a protrusion 520 or other coding feature that corresponds to the groove 320 or other coding feature within cartridge holder 300. Thus, when a user inserts dose setting mechanism 500 into cartridge holder 300, the protrusion 520 slides within groove 320.

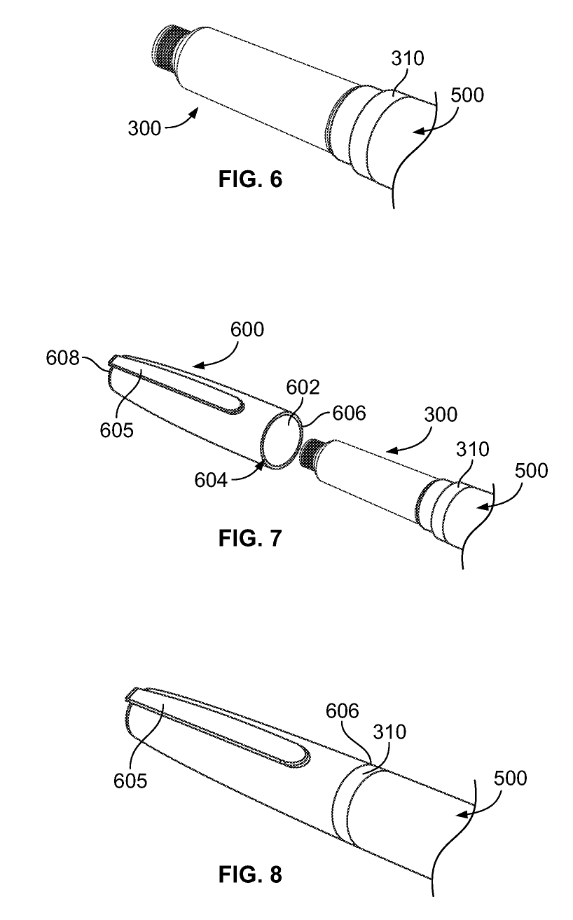

FIG. 6 illustrates the drug delivery device from FIG. 5, comprising the cartridge holder, cartridge (not shown), and dose setting mechanism, in an assembled position. As is shown in FIG. 6, color-coded ring 310 remains visible after the cartridge holder 300 is affixed to the dose setting mechanism 500. Thus, a user needing to take a certain drug can easily see color-coded ring 310 and know which drug is contained within the drug delivery device.

FIG. 7 illustrates the drug delivery device from FIG. 6 with a cap 600 being placed over cartridge holder 300. Cap 600 comprises a proximal end 606, a distal end 608, and an interior surface 602 defining a cap aperture 604. Cap aperture 604 is placed over distal end 306 of cartridge holder 300. Cap 600 may also comprise a differentiation feature such as a designated color on a clip 605 or in another location. The designated color may be selected to correspond or match color-coded ring 310. A user will know which cap corresponds with which cartridge assembly by matching up the colors on clip 605 and color-coded ring 310. In addition, cap 600 may also comprise mechanical and/or electrical coding to help ensure that the cap 600 can only be fitted on the correct cartridge holder or correct dose setting mechanism.

FIG. 8 illustrates the drug delivery device from FIG. 7 with the cap fully fitted over the cartridge holder. As can be seen in FIG. 8, even when cap 600 is secured and covers cartridge holder 300, color-coded ring 310 remains exposed and visible. This is because proximal end 606 of cap 600 abuts an end of color-coded ring 310. Thus, a user can plainly see that the color on clip 605 matches the color on color-coded ring 310.

One advantage of using the dose setting mechanism 300 with a color ring and other differentiating features is that a user can easily differentiate various drug delivery devices and determine which drug is contained within which drug delivery device. In addition, during an assembly stage, these differentiating features can be used to help ensure that only a proper cartridge is placed within their corresponding cartridge holders and are assembled with the other various correct pieces. That is, the differentiating features help to prevent both unwanted attachment of non-corresponding parts of a drug delivery device, as well as the accidental delivery of an incorrect drug by a user.

Those of skill in the art will recognize alternative geometries of the color-coded ring may also be used. For example, the geometric shape or the overall size of the ring may be altered. In addition or as an alternative to the color-coding, words, graphics, or images may also be present on the ring, for example, stating the name of the drug within the cartridge to be used.

The proposed differentiating features may apply to any drug delivery device, with any type of reservoir or primary pack, e.g. inhaler, pouch.

The disclosed proposed differentiating features result in a number of advantages. For example, the color-coded ring with coding features helps to ensure that a delivery device can only be used with a medicament for which the device is intended. The disclosed differentiating features help prevent a user from affixing a dose setting mechanism to the wrong cartridge assembly.

The disclosed differentiating features also result in a low cost drug delivery device since the features do not require a large number of parts and can be manufactured in a cost effective manner. In particular, the same dispensing mechanism may be used for a wide range or medicaments, and differentiation is applied only to a small number of parts.

Exemplary embodiments have been described. Those skilled in the art will understand, however, that changes and modifications may be made to these arrangements without departing from the true scope and spirit of the present disclosure, which is defined by the claims.

1-17. (canceled)

18: An assembly comprising:
a cartridge and a cartridge holder, wherein the cartridge is provided with a differentiation feature, wherein said differentiation feature is arranged to remain visible when said cartridge is inside the cartridge holder and the cartridge or the cartridge holder is assembled to a dose setting mechanism of a drug delivery device, wherein a main body is affixed to a proximal end or near the proximal end of said cartridge, and wherein the differentiation feature is provided along said main body, characterized in that said main body comprises a color-coded ring or collar, wherein said color-coded ring or collar wraps around an exterior circumference of said cartridge.

19: The assembly of claim 18, wherein the differentiation feature is arranged near or at the proximal end of the cartridge.

20: The assembly of claim 18, wherein the differentiation feature comprises a color.

21: The assembly of claim 18 further comprising a cap for covering at least a distal end of said cartridge or said cartridge holder, wherein when said cap is removably affixed to said cartridge or cartridge holder, said differentiation feature remains visible.
